(19) United States
(12) Patent Application Publication
(10) Pub. No.: US 2007/0071699 A1
(43) Pub. Date: Mar. 29, 2007

(54) Benzylidene-1,3-thiazolidine-2,4-dione compounds for promoting and/or inducing and/or stimulating the pigmentation of keratin materials and/or for limiting their depigmentation and/or whitening

(75) Inventor: Christophe Boulle, Paris (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/476,051

(22) Filed: Jun. 28, 2006

Related U.S. Application Data
(60) Provisional application No. 60/697,975, filed on Jul. 12, 2005.

(30) Foreign Application Priority Data
Jun. 28, 2005 (FR) 05/51791

(51) Int. Cl. A61K 8/49; A61K 31/426

(52) U.S. Cl. 424/59; 514/369

(57) ABSTRACT

The benzylidene-1,3-thiazolidine-2,4-dione compounds having the structural formula (I), or salt and/or solvate and/or isomer thereof: 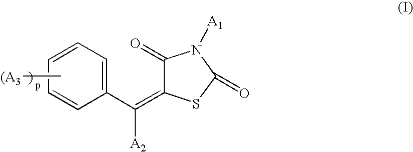 are useful active agents for promoting and/or inducing and/or stimulating the pigmentation of keratin materials and/or preventing and/or limiting the depigmentation and/or bleaching thereof, particularly of human head hair, beard hair, moustache hair, eyelashes and eyebrows, and advantageously are active agents for preventing and/or limiting the canities of human keratin fibers.

### CROSS-REFERENCE TO PRIORITY/PROVISIONAL APPLICATIONS

This application claims priority under 35 U.S.C. §119 of FR 05/51791, filed Jun. 28, 2005, and of U.S. Provisional Application No. 60/697,975, filed Jul. 12, 2005, each hereby expressly incorporated by reference and each assigned to the assignee hereof.

### CROSS-REFERENCE TO COMPANION APPLICATIONS

Copending applications Ser. No. ______ [Attorney Docket No. 1016800-000769], Ser. No. ______ [Attorney Docket No. 1016800-000770], and Ser. No. ______ [Attorney Docket No. 1016800-000772], filed concurrently herewith, each hereby also expressly incorporated by reference and each also assigned to the assignee hereof.

### BACKGROUND OF THE INVENTION

1. Technical Field of the Invention

The present invention relates to the cosmetic/therapeutic administration of at least one benzylidene-1,3-thiazolidine-2,4-dione compound of particular formula as an active agent for promoting and/or inducing and/or stimulating the pigmentation of keratin materials and/or for limiting their depigmentation and/or whitening, and more particularly as a bioactive agent for preventing and/or limiting the canities of human keratin fibers.

The keratin materials to which this invention pertains include human skin and nails, and human keratin fibers such as the hair, the eyebrows, the eyelashes, beard hair and moustache hair. The present invention also applies to the keratin materials of mammalian animals (for example dogs, horses or cats). More especially, this invention pertains to human head hair, beard hair, moustache hair, eyelashes and eyebrows.

2. Description of Background and/or Related and/or Prior Art

Need exists for novel products for caring for and/or treating human keratin materials that promote their pigmentation and/or limits their depigmentation, and more particularly for products that can prevent and/or reduce the canities of human keratin fibers such as the hair, the eyelashes and/or certain bodily hairs.

The color of human hair and skin depends on various factors and especially on the seasons of the year, race, sex and age. It is mainly determined by the concentration of melanin produced by the melanocytes. These melanocytes are specialized cells that synthesize melanin via particular organelles, the melanosomes.

Melanin synthesis (or melanogenesis) is complex and schematically involves the following principal steps:

Tyrosine→Dopa→Dopaquinone→Dopachrome→Melanin

Tyrosinase (monophenol dihydroxyl phenylalanine: oxygen oxidoreductase EC 1.14.18.1) participates in this sequence of reactions by especially catalyzing the reaction for conversion of tyrosine into dopa (dihydroxyphenylalanine) and the reaction for conversion of dopa into dopaquinone.

The upper part of the hair follicle appears as a tubular invagination of the epidermis, which is buried just down to the deep layers of the dermis. The lower part, or hair bulb, itself comprises an invagination in which is found the dermal papilla. Around the dermal papilla, in the lower part of the bulb, is an area populated with cells with a high degree of proliferation (matrix cells). These cells are the precursors of the keratinized cells that will constitute the hair. The cells that result from the proliferation of these precursors migrate vertically in the bulb and become gradually keratinized in the upper part of the bulb; this assembly of keratinized cells will form the hair stem. Pigmentation of the hair and of other bodily hairs requires the presence of melanocytes in the bulb of the hair follicle. These melanocytes are in an active state, i.e., they synthesize melanins (or melanin pigments). These pigments are transferred to the keratinocytes intended to form the hair stem, which will give rise to the growth of a pigmented head hair or other bodily hair. This structure is known as a “follicular pigmentation unit”.

It is known that, in the majority of populations, a brown skin coloration and maintenance of a constant coloration of head hair are important aspirations.

It is accepted that the appearance of grey or white bodily hairs and/or head hairs, or canities, is associated with a decrease in melanin in the hair stem. This phenomenon occurs naturally during the life of an individual. However, individuals are seeking to have a more youthful appearance and, with an aesthetic aim, they are often tempted to combat this phenomenon, especially when it occurs at a relatively early age.

Many solutions have thus been proposed in the field of artificial coloration by providing exogenous dyes intended to give the hair a coloration as close as possible to that which it has naturally. Another approach consists in stimulating the natural pigmentation pathway.

Among the proposed solutions, exemplary are the compositions containing a phosphodiesterase inhibitor (WO 95/17161), DNA fragments (WO 95/01773), diacylglycerol (WO 94/04122), prostaglandins (WO 95/11003) or pyrimidine 3-oxide derivatives (EP 829 260).

It has now unexpectedly been found that it is possible to stimulate the synthesis of melanin by the melanocytes by specifically inhibiting the degradation of the prostaglandins synthesized by these melanocytes or those present in its environment.

The involvement of certain prostaglandins in the pigmentation of bodily hairs or the skin in man or animals is described in the document Wand M., 1997, Arch. Ophtalmol., 115; Abdel Malek et al., 1987, Cancer Res., 47. However, since prostaglandins are molecules with a very short biological half-life and as a result of the local and labile nature of their metabolism (Narumiya S. et al.), it appears important to be able to prolong the activity of the prostaglandins involved in the pigmentation of human skin, bodily hairs and/or head hair.

In WO 04/073 594, the assignee hereof has shown that 15-hydroxyprostaglandin dehydrogenase (15-PGDH) is also expressed in the hair melanocyte, which has never been demonstrated hitherto. In addition, the assignee hereof has demonstrated the presence of 15-PGDH in the dermal papilla and the melanocyte of head hairs, and has proposed administering a 15-PGDH inhibitor to promote the pigmentation of human skin, bodily hairs and/or head hair. It is now possible to locally regulate the level of prostaglandins and especially that present in the melanocyte, in particular of head hair, by acting on the degradation catalyzed both by the 15-PGDH of the melanocyte and of the fibroblast of the dermal papilla.

Type-1 15-PGDH is a key enzyme in the deactivation of prostaglandins, in particular of PGF2-α and PGE2, which are important mediators of the growth and survival of the hair. It corresponds to the classification EC 1.1.1.141 and is NAD⁺-dependent. This enzyme catalyzes an oxidation reaction of the hydroxyl in position 15 to ketone. It has been isolated from pig kidney; its inhibition has especially been observed with a thyroid hormone, triiodothyronine, at doses very much higher than the physiological doses. Type-2 15-PGDH is itself NADP-dependent.

In this same patent application, it has also been shown that the hair melanocytes express prostaglandin H synthase 1 (PGHS-1 or COX-1, E.C.:1.14.99.1). This demonstrates for the first time that the hair melanocytes have an autonomous prostaglandin metabolism.

In WO 04/073594, it has moreover been shown that it is possible to specifically inhibit the 15-PGDH present in the dermal papilla and/or in the hair melanocyte. Such an inhibition thus makes it possible to stop the deactivation of the prostaglandins in the environment of the hair melanocyte. The prostaglandins can thus continue, via an autocrine or paracrine route, to stimulate the melanocytes. In point of fact, the application of such inhibitors stimulates the production of melanin by the melanocytes.

### SUMMARY OF THE INVENTION

According to the invention, the term “15-PGDH inhibitor” means any substance, a simple or complex compound, of natural or synthetic origin, capable of inhibiting or reducing the activity of the enzyme 15-PGDH, and/or capable of inhibiting, reducing or slowing down the reaction catalyzed by this enzyme. The 15-PGDH inhibitors according to the invention are preferably inhibitors of type-1 15-PGDH.

Advantageously, the inhibitor is a specific inhibitor of the NAD-dependent type-1 15-PGDH.

In addition, it has now surprisingly been found that certain benzylidene-1,3-thiazolidine-2,4-dione compounds of formula (I) that will be defined in detail hereinbelow, are inhibitors of 15-hydroxyprostaglandin dehydrogenase, in particular of type 1. It has moreover been found that these same benzyl-1,3-thiazolidine-2,4-dione or benzylidene-1,3-thiazolidine-2,4-dione compounds make it possible to promote and/or induce and/or stimulate the pigmentation of keratin materials, and also to limit their depigmentation and/or whitening.

The present invention thus features the cosmetic/therapeutic administration of at least one benzylidene-1,3-thiazolidine-2,4-dione compound of formula (I) or a salt and/or solvate and/or cis-trans or Z/E isomer thereof, as an active agent for promoting and/or inducing and/or stimulating the pigmentation of keratin materials and/or as an active agent for preventing and/or limiting the depigmentation and/or whitening of keratin materials and more particularly of human keratin fibers, for instance human head hair, beard hair, moustache hair, eyelashes and eyebrows.

The present invention more particularly features the cosmetic/therapeutic administration of at least one benzylidene-1,3-thiazolidine-2,4-dione compound of formula (I), or a salt and/or solvate and/or cis-trans or Z/E isomer thereof, as an active agent for preventing and/or limiting the canities of human keratin fibers.

The present invention also features the cosmetic/therapeutic formulation of at least one benzylidene-1,3-thiazolidine-2,4-dione compound of formula (I), or a salt and/or solvate and/or cis-trans or Z/E isomer thereof, in a care and/or makeup composition for inducing and/or stimulating the pigmentation of keratin materials and/or for limiting their depigmentation and/or whitening, and more particularly of human keratin fibers such as human head hair, beard hair, moustache hair, eyelashes and eyebrows.

This invention more particularly features the cosmetic/therapeutic formulation of at least one benzylidene-1,3-thiazolidine-2,4-dione compound of formula (I), or of a salt and/or solvate and/or cis-trans isomer thereof, in a care and/or makeup composition for preventing and/or limiting the canities of human keratin fibers.

The present invention also features the cosmetic use of at least one benzylidene-1,3-thiazolidine-2,4-dione compound of formula (I), or a salt and/or solvate and/or cis-trans or Z/E isomer thereof, in the manufacture of a composition for inducing and/or stimulating the pigmentation of keratin materials and/or for limiting their depigmentation and/or whitening, and more particularly of human keratin fibers such as human head hair, beard hair, moustache hair, eyelashes and eyebrows.

The present invention more particularly features the use of at least one benzylidene-1,3-thiazolidine-2,4-dione compound of formula (I), or a salt and/or solvate and/or cis-trans or 2/E isomer thereof, in the manufacture of a composition for preventing and/or limiting the canities of human keratin fibers such as human head hair, beard hair, moustache hair, eyelashes and eyebrows.

The present invention also features a cosmetic/therapeutic regime or regimen for inducing and/or stimulating the pigmentation of keratin materials and more particularly of human keratin fibers, and/or for limiting their depigmentation and/or whitening, comprising topically applying to the said keratin materials an effective amount of at least one benzylidene-1,3-thiazolidine-2,4-dione compound of formula (I), or a salt and/or solvate and/or cis-trans or Z/E isomer thereof.

The present invention also features a cosmetic regime or regimen for treating the canities of human keratin fibers, in particular the hair, beard hair, moustache hair, the eyelashes and/or the eyebrows, comprising topically applying to the said fibers an effective amount of at least one benzylidene-1,3-thiazolidine-2,4-dione compound of formula (I), or a salt and/or solvate and/or cis-trans or Z/E isomer thereof.

The benzylidene-1,3-thiazolidine-2,4-dione compounds of the invention or the salts and/or solvates and/or cis-trans or Z/E isomers thereof have the following formula (I): 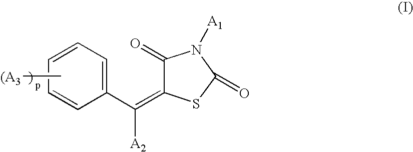
in which:
a) A₁ is:
   1) a hydrogen atom; or
   2) a linear or branched, saturated or unsaturated C₁-C₂₀ alkyl radical optionally substituted with one or more groups Z₁;
b) A₂ and A₃ independently are:
   1) a hydrogen atom;
   2) a linear or branched, saturated or unsaturated C₁-C₂₀ alkyl radical optionally substituted with one or more groups Z₁;
   3) a group Z₁;
   with the provisos that when A₃ is Z₁, then
   (i) Z₁ is other than chlorine; and
   (ii) when Z₁ is OZ₂, then Z₂ is other than hydrogen, a 2-chloro-6-fluorobenzyl group, a 1,1,3,3-tetramethylbutylphenyl group and a 2,4-di(tert-butyl)phenyl group;
c) p ranges from 0 to 5 inclusive and, when n>1, the substituents A₅ may be identical or different;
d) Z₁ is:
   1) a halogen, for instance F, Cl or Br;
   2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂COZ′₂, NZ₂CONZ′₂Z″₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ and Si(OZ₂)(OZ′₂)OZ″₂;
e) the radicals Z₂, Z′₂, Z″₂ and Z′″₂ independently are:
   1) a hydrogen atom;
   2) a linear or branched, saturated or unsaturated C₁-C₂₀ alkyl radical optionally substituted with one or more groups Z₃; or
   3) a saturated or unsaturated ring member of 4 to 15 atoms, optionally containing at least one heteroatom selected from among O, N and S, optionally fused to another ring, these rings being optionally substituted with one or more groups Z₃; this ring is advantageously phenyl;
f) the radical Z₃ is:
   1) a group Z₄;
   2) a linear or branched, saturated or unsaturated C₁-C₂₀ alkyl radical optionally substituted with one or more groups Z₄; or
   3) a saturated or unsaturated ring member of 4 to 15 atoms, optionally containing at least one heteroatom selected from among O, N and S, optionally fused to another ring, these rings being optionally substituted with one or more groups Z₄;
g) Z₄ is:
   1) a halogen atom, for instance F, Cl or Br; or
   2) a group selected from among CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅N′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ and Si(OZ₅)(OZ′₅)OZ″₅;
h) the radicals Z₅, Z′₅, Z″₅ and Z′″₅ independently are:
   1) a hydrogen atom;
   2) a linear or branched, saturated or unsaturated C₁-C₂₀ alkyl radical; or
   3) a saturated or unsaturated ring member of 4 to 15 atoms, optionally containing at least one heteroatom selected from among O, N and S, optionally fused to another ring, these rings being optionally substituted with one or more CF₃, halogen or linear or branched C₁-C₂₀ alkyl radicals.

### DETAILED DESCRIPTION OF BEST MODE AND SPECIFIC/PREFERRED EMBODIMENTS OF THE INVENTION

According to the invention, the term “salts of the compound of formula (I)” means the organic or mineral salts of a compound of formula (I).

As mineral salts according to the invention, exemplary are the sodium or potassium salts and also the ammonium, zinc (Zn²⁺), calcium (Ca²⁺), copper (Cu²⁺), iron (Fe²⁺ and Fe³⁺), strontium (Sr²⁺), magnesium (Mg²⁺) and manganese (Mn²⁺) salts; hydroxides, hydrohalides (for example hydrochlorides), carbonates, hydrogen carbonates, sulfates, hydrogen phosphates, phosphates.

The organic salts according to the invention are, for example, the triethanolamine, monoethanolamine, ethanolamine, hexadecylamine and N,N,N′,N′-tetrakis(2-hydroxypropyl)ethylenediamine salts, and also organic acid salts, for instance citrates, lactates, glycolates, gluconates, acetates, propionates, fumarates, oxalates and tartrates.

As possible solvates of the compounds of formula (I), exemplary are the hydrates, alcoholates and hydroalcoholates.

According to the invention, the compounds of formula (I) are in isolated form, i.e., non-polymeric form.

According to the invention, the term “at least one” means one or more (2, 3 or more). In particular, the composition may contain one or more compounds of formula (I). This or these compound(s) may be cis or trans or Z or E isomers or a mixture of cis/trans or Z/E isomers. They may also be in tautomeric form. This or these compound(s) may be enantiomers and/or diastereoisomers or a mixture of these isomers, in particular a racemic mixture.

For the purposes of the invention, the term “alkyl radical” means a hydrocarbon-based radical that may be linear or branched and saturated or unsaturated. In particular, the alkyl radical contains from 1 to 20 and preferably from 1 to 10 carbon atoms. As examples of alkyl radicals according to the invention, mention may be made of methyl, ethyl, isopropyl, n-butyl, tert-butyl, n-hexyl, 2-ethylhexyl, ethylene and propylene radicals.

As halogen atoms according to the invention, mention may be made of chlorine, fluorine or bromine atoms, and better still chlorine and fluorine atoms.

As saturated rings according to the invention, mention may be made of cyclopropyl, cyclobutyl, cyclopentyl, cyclohexyl, cycloheptyl, admantyl, etc. radicals.

Unsaturated rings that are exemplary include cyclohexenyl and phenyl radicals. As fused hydrocarbon-based rings according to the invention, exemplary are naphthyl and azulenyl radicals.

As fused rings of different nature according to the invention, exemplary are benzofuran, dibenzofuran, benzothiophene, benzothiazole, indole, benzimidazole, quinoline, isoquinoline, quinazoline, chromene, carbazole and fluorene rings.

As examples of rings containing a carbonyl or thiocarbonyl function according to the invention, mention may be made of the following rings:

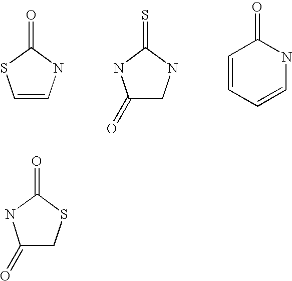

As examples of heterocycles according to the invention, mention may be made, independently, of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine, diazepine or 15-C-5 crown ether rings. Pyrrole, pyrrolidine, imidazole, furan, thiophene, oxazole, thiazole, isoxazole, isothiazole, oxadiazole, pyrazole, tetrazole, pyridine, pyrimidine, triazole, pyrazine, pyridazine, piperidine, piperazine and morpholine rings are preferably used.

Among the compounds of formula (I) in accordance with the invention that are more particularly preferred are those selected from among the following list of compounds:

| Com- | |
|---|---|
| pound | Structure |
| 1 | 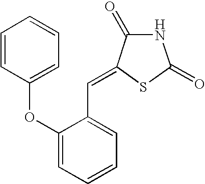 |
| 2 | 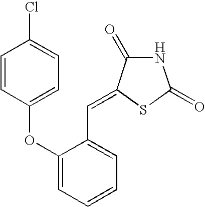 |
| 3 | 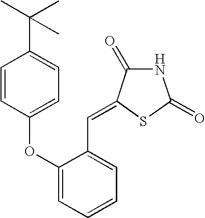 |
| 4 | 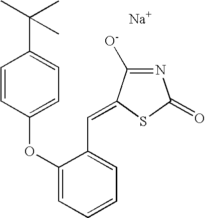 |
| 5 | 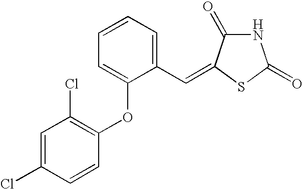 |
| 6 | 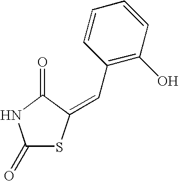 |
| 7 | 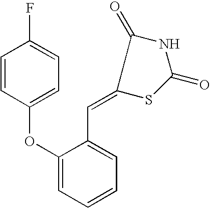 |
| 8 | 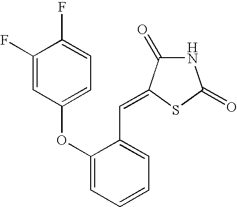 |
| 9 | 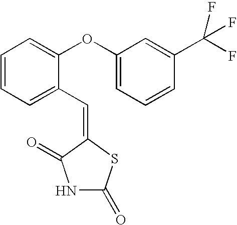 |
| 10 | 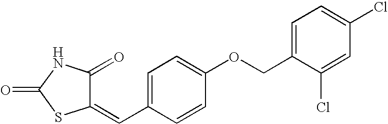 |
| 11 | 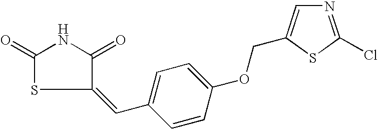 |
| 12 | 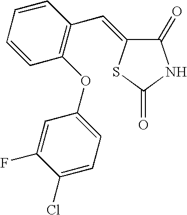 |
| 13 | 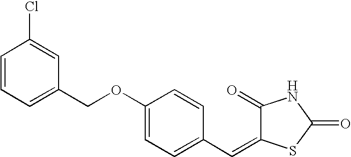 |
| 14 | 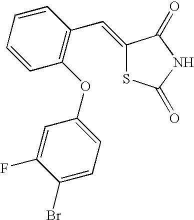 |
| 15 | 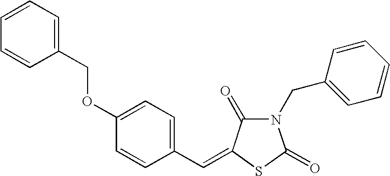 |
| 16 | 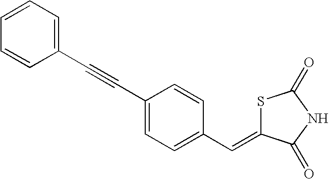 |
| 17 | 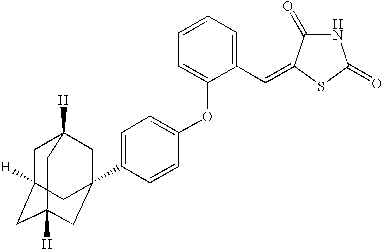 |
| 18 | 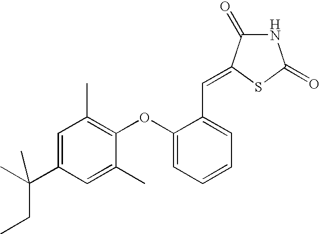 |
| 19 | 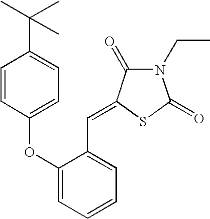 |
| 20 | 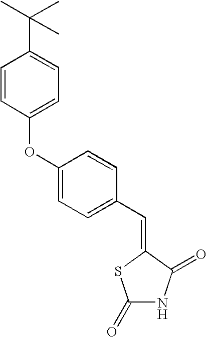 |

The compounds of formula (I) are preferably selected from among those for which the following conditions are satisfied:

A₁ and A₂ simultaneously are hydrogen;

A₃ is a group Z₁;

Z₁ is a group OZ₂ in which Z₂ is a phenyl group optionally substituted with one or more adamantyl, CF₃, halogen or linear or branched, saturated or unsaturated C₁-C₂₀ alkyl radicals.

The compounds that are more particularly preferred are selected from among:

(1) the compound (5Z)-5-[2-(3,4-difluorophenoxy)benzylidene]-1,3-thiazolidine-2,4-dione of structure:

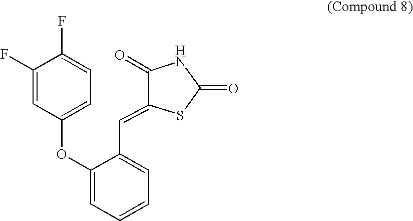

(2) the compound (5Z)-5-[2-(4-tert-butylphenoxy)benzylidene]-1,3-thiazolidine-2,4-dione of structure:

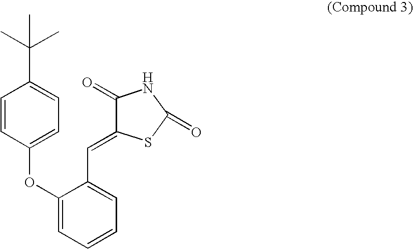

(3) its sodium salt of structure of structure:

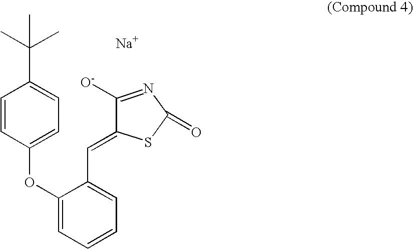

(5) the compound (5Z)-5-[2-(4-chloro-3-fluorophenoxy)benzylidene]-1,3-thiazolidine-2,4-dione of structure:

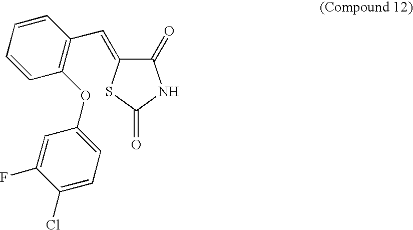

(6) the compound (5E)-5-[4-[(3-chlorobenzyl)oxy]benzylidene}-1,3-thiazolidine-2,4-dione of structure:

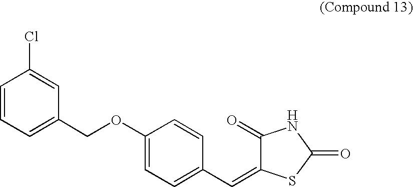

(7) the compound (5Z)-5-[4-(4-tert-butylphenoxy)benzylidene]-1,3-thiazolidine-2,4-dione of structure:

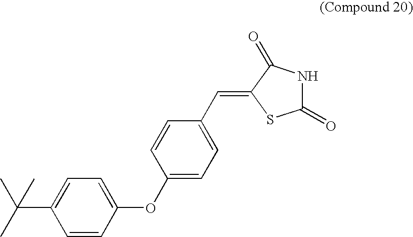

The compounds of formula (I) in accordance with the invention may be obtained according to a synthetic route as described in the reaction scheme defined below. The synthetic route used entails an aromatic nucleophilic substitution, then a Knoevenagel condensation:

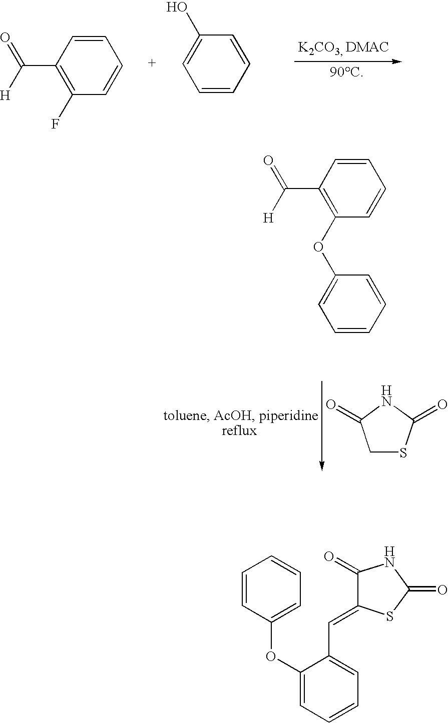

The effective amount of compound of formula (I) (salified or non-salified, solvated or non-solvated) corresponds to the amount necessary to obtain the desired result (namely induce or stimulate keratin pigmentation, especially of the hair and the eyelashes, and/or to reduce their depigmentation and/or whitening). One skilled in this art is therefore capable of evaluating this effective amount, which depends on the nature of the compound used, on the individual to whom it is applied and on the length of time of this application.

In the text hereinbelow, and unless otherwise indicated, the amounts of the various ingredients in the composition are given as weight percentages relative to the total weight of the composition.

To provide an order of magnitude, according to the invention, the compound of formula (I) (salified or non-salified, and solvated or non-solvated) or a mixture of compounds of formula (I) (salified or non-salified, and solvated or non-solvated) may be used in an amount ranging from 10⁻³% to 10% of the total weight of the composition, preferably in an amount ranging from 10⁻³% to 5% and better still from 10⁻²% to 2% of the total weight of the composition, for example from 0.5% to 2%.

The compositions of the invention may be for cosmetic or pharmaceutical application. The compositions of the invention are preferably for cosmetic application. In addition, the composition must contain a non-toxic, physiologically acceptable medium that can be applied to human skin, including the scalp and the eyelids, and to human keratin fibers. For the purposes of the invention, the term “cosmetic” means a composition of pleasant appearance, odor and feel.

The compounds of formula (I) (salified or non-salified, and solvated or non-solvated) may be formulated into a composition that should be ingested, injected or applied to the skin or to keratin fibers (to any area of skin or fibers to be treated).

According to the invention, the compound of formula (I) or a mixture of compounds of formula (I) may be administered orally in an amount of from 0.1 mg to 300 mg per day, for example from 5 mg/day to 10 mg/day.

A preferred composition of the invention is a composition for cosmetic use and in particular for topical application to the skin and keratin fibers, and more especially to the scalp, the hair and the eyelashes.

This composition may be in any known presentation form that is suitable for the mode of use.

For topical application to the skin or keratin fibers, the composition may be in the form of an aqueous, alcoholic or aqueous-alcoholic solution or suspension, or an oily suspension or solution, an emulsion or dispersion of more or less fluid consistency and especially of liquid or semi-liquid consistency, obtained by dispersion of a fatty phase in an aqueous phase (O/W) or conversely (W/O), a solid (O/W) or (W/O) emulsion or dispersion, a more or less fluid or solid aqueous, aqueous-alcoholic or oily gel, a free or compacted powder to be used in unmodified form or to be incorporated into a physiologically acceptable medium, or alternatively microcapsules, microparticles or vesicular dispersions of ionic and/or nonionic type.

A composition in the form of a foam or alternatively in the form of a spray or aerosol, then comprising a pressurized propellant, may also be envisaged.

It may thus be in the form of a lotion, serum, milk, O/W or W/O cream, gel, unguent, ointment, powder, balm, patch, impregnated pad, cake or foam.

In particular, the composition for application to the scalp or the hair may be in the form of a haircare lotion, for example for daily or twice-weekly application, a shampoo or a hair conditioner, in particular for twice-weekly or weekly application, a liquid or solid scalp cleansing soap for daily application, a hairstyle shaping product (lacquer, hair setting product or styling gel), a treatment mask, a foaming gel or cream for cleansing the hair. It may also be in the form of a hair dye or mascara to be applied with a brush or a comb.

Moreover, for application to the eyelashes or body hairs, the composition to which the invention applies may be in the form of a pigmented or unpigmented mascara, to be applied with a brush to the eyelashes or alternatively to beard or moustache hair.

For a composition for use by injection, the composition may be in the form of an aqueous lotion or an oily suspension. For oral use, the composition may be in the form of capsules, granules, drinkable syrups or tablets.

According to one particular embodiment, the composition according to the invention is in the form of a hair cream or hair lotion, a shampoo, a hair conditioner, a hair mascara or an eyelash mascara.

The amounts of the various constituents of the physiological medium of the composition according to the invention are those generally employed in the fields under consideration. In addition, these compositions are prepared according to the usual methods.

When the composition is an emulsion, the proportion of the fatty phase may range from 2% to 80% by weight and preferably from 5% to 50% by weight relative to the total weight of the composition. The aqueous phase is adjusted as a function of the content of fatty phase and of compound(s) (I) and also of that of the optional additional ingredients, to obtain 100% by weight. In practice, the aqueous phase represents from 5% to 99.9% by weight.

The fatty phase may contain fatty or oily compounds that are liquid at room temperature (25° C.) and atmospheric pressure (760 mm Hg), which are generally known as oils. These oils may be mutually compatible or incompatible and may form a macroscopically homogeneous liquid fatty phase or a two-phase or three-phase system.

In addition to the oils, the fatty phase may contain waxes, gums, lipophilic polymers or “pasty” or viscous products containing solid parts and liquid parts.

The aqueous phase contains water and optionally an ingredient that is miscible in all proportions with water, for instance C₁ to C₈ lower alcohols such as ethanol or isopropanol, polyols, for instance propylene glycol, glycerol or sorbitol, or alternatively acetone or ether.

For a composition in emulsion form, the composition may contain one or more emulsifiers optionally combined with one or more co-emulsifiers used to obtain a composition in emulsion form; these emulsifiers and co-emulsifiers are those generally used in cosmetics and pharmaceuticals. Their nature also depends on the sense of the emulsion. In practice, the emulsifier and, where appropriate, the co-emulsifier are present in the composition in a proportion ranging from 0.1% to 30% by weight, preferably from 0.5% to 20% by weight and better still from 1% to 8% by weight. The emulsion may also contain microcapsules or microparticles, and vesicular dispersions and especially lipid vesicles such as liposomes.

When the composition is in the form of an oily solution or gel, the fatty phase may represent more than 90% of the total weight of the composition.

Advantageously, for a hair application, the composition of the invention is an aqueous, alcoholic or aqueous-alcoholic solution or suspension and better still a water/ethanol solution or suspension. The alcoholic fraction may represent from 5% to 99.9% and better still from 8% to 80%.

For a mascara application, the composition of the invention is especially in the form of a wax-in-water or wax-in-oil dispersion, a gelled oil or an aqueous gel, which may be pigmented or unpigmented.

The compositions of the invention may also comprise other additional ingredients usually employed in the fields under consideration, selected from among solvents, aqueous-phase or oily-phase thickeners or gelling agents, dyestuffs that are soluble in the medium of the composition, solid particles such as fillers or pigments, antioxidants, sequestrants, preservatives, fragrances, electrolytes, neutralizers, film-forming polymers, UV blockers, for instance sunscreens, cosmetic and pharmaceutical active agents with a beneficial effect on the skin or keratin fibers, other than the compounds of formula (I), and mixtures thereof. These additives may be present in the composition in the amounts generally used in cosmetics and dermatology, and especially in a proportion of from 0.01% to 50% and better still from 0.1% to 20%, for example from 0.1% to 10%, relative to the total weight of the composition. Depending on their nature, these additives may be introduced into the fatty phase, into the aqueous phase and/or into the lipid vesicles and especially liposomes.

Needless to say, one skilled in the art will take care to select the optional additional ingredients and/or the amount thereof such that the advantageous properties of the compositions according to the invention, i.e., the inhibition of 15-PGDH and in particular the increase in the density of keratin fibers and/or the reduction of their whitening are not, or are not substantially, adversely affected by the envisaged addition.

As solvents according to the invention, exemplary are C₂ to C₈ lower alcohols, for instance ethanol, isopropanol, propylene glycol and certain light cosmetic oils, for instance C₆ to C₁₆ alkanes and also benzyl alcohol.

As oils that may be used in the invention, exemplary are oils of mineral origin (liquid petroleum jelly or hydrogenated isoparaffin), oils of plant origin (liquid fraction of shea butter, sunflower oil, apricot oil, fatty alcohol or fatty acid), oils of animal origin (perhydrosqualene), synthetic oils (fatty acid ester, purcellin oil), silicone oils (linear or cyclic polydimethylsiloxane, phenyl trimethicone) and fluoro oils (perfluoropolyethers). Waxes that may be mentioned include silicone waxes, beeswax, rice wax, candelilla wax, carnauba wax, paraffin wax and polyethylene wax.

As emulsifiers according to the invention, examples include glyceryl stearate, glyceryl laurate, sorbitol stearates, sorbitol oleates, alkyl dimethicone copolyols (with alkyl≧8) and mixtures thereof for a W/O emulsion. Polyethylene glycol monostearate or monolaurate, polyoxyethylenated sorbitol stearate or oleate, and dimethicone copolyols, and mixtures thereof, may also be used for an O/W emulsion.

As hydrophilic gelling agents according to the invention, exemplary are carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, mention may be made of modified clays, for instance Bentones, metal salts of fatty acids, for instance aluminum stearates, hydrophobic-treated silica and ethylcellulose, and mixtures thereof.

As cosmetic or pharmaceutical active agent other than the compounds of formula (I) according to the invention, exemplary are hydrophilic active agents such as proteins or protein hydrolysates, amino acids, polyols, urea, allantoin, sugars and sugar derivatives, water-soluble vitamins, plant extracts (those from Iridacea plants or from soybean) and hydroxy acids such as fruit acids or salicylic acid; and lipophilic active agents such as retinol (vitamin A) and its derivatives, especially an ester (retinyl palmitate), tocopherol (vitamin E) and its derivatives, especially an ester (tocopheryl acetate), essential fatty acids, ceramides, essential oils, salicylic acid derivatives, for instance 5-n-octanoylsalicylic acid, hydroxy acid esters, and phospholipids, for instance lecithin, and mixtures thereof.

Advantageously, the compositions according to the invention also comprise at least one 15-PGDH inhibitor as defined above and at least one prostaglandin or prostaglandin derivative, for instance the prostaglandins of series 2 especially including PGF2-α and PGE2 in salt or ester form (for example the isopropyl esters), derivatives thereof, for instance 16,16-dimethyl PGE2, 17-phenyl PGE2,16,16-dimethyl PGF2-α, 17-phenyl PGF2-α, prostaglandins of series 1, for instance 11-deoxyprostaglandin E1, 11-deoxyprostaglandin E1 in salt or ester form, analogues thereof, especially latanoprost, (5E)-7-{(1R,2R,3R,5S)-3,5-dihydroxy-2-[(3R)-3-hydroxy-5-phenylpentyl]cyclopentyl}hept-5-enoic acid, viprostol, bimatoprost, cloprostenol, travoprost, fluprostenol, cloprostenol, butaprost, unoprostone, misoprostol, and the salts or esters thereof.

The compositions comprising at least the compound of formula (I), salified or non-salified, solvated or non-solvated, may be encapsulated in particular in liposomal form, as described especially in WO 94/22468. Thus, the encapsulated compound may be delivered selectively to the hair follicle.

The compositions according to the invention may be applied in a regime or regimen to the areas of the skin to be treated and in particular to the alopecic areas of the scalp and hair of an individual, or only to the white hairs, and optionally left in contact for several hours and optionally rinsed off.

The compositions containing an effective amount of a compound of formula (I), salified or non-salified, solvated or non-solvated, may, for example, be applied in the evening, kept in contact throughout the night and optionally shampooed out in the morning. These applications may be repeated daily for one or more months according to the individual.

Advantageously, in the regime or regimen according to the invention, between 5 μL and 10 ml of a solution or composition as defined above, comprising from 0.001% to 5% of 15-PGDH inhibitor, are applied to the areas of the scalp and/or the hair to be cared for or treated.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

Synthesis of the compound (5Z)-5-[2-(4-tert-butylphenoxy)benzylidene]-1,3-thiazolidine-2,4-dione of formula (I) (A₁=A₂=H; A₃=OZ₂; Z₂ is phenyl substituted with a tert-butyl group):

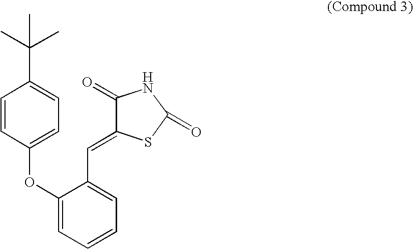

2-[4-(tert-butyl)phenoxy]benzaldehyde (10 g; 39.3×10⁻³ mol) is diluted in 120 mL of toluene in a 250 mL round-bottomed flask on which is mounted Dean-Stark apparatus and a condenser, followed by addition of 2,4-thiazolidinedione (4.6 g; 39.9×10⁻³ mol), piperidine (1 mL) and acetic acid (1 mL). The reaction medium is refluxed for 4 hours. After cooling to room temperature, a precipitate forms, which is filtered off and then rinsed several times with toluene. The pale yellow solid obtained is dried under vacuum at 50° C. to give 8.54 g of product (yield: 62%).

### EXAMPLE 2

### Demonstration of the 15-PGDH-Specific Inhibitory Properties of the Compounds of Formula (I)

Test on type-1 15-PGDH:

The enzyme 15-PGDH is obtained as described in FR 02/05067 filed in the name of L'Oréal, as a suspension in a medium adjusted to a concentration of 0.3 mg/mL and then blocked at −80° C. For the purposes of the test, this suspension is thawed and stored in ice.

In parallel, a 100 mM, pH 7.4 Tris buffer containing 0.1 mM of dithiothreitol (D5545, Sigma-Aldrich, L'isle D'Abeau Chesne, BP 701, 38297, Saint Quentin Fallavier), 1.5 mM of β-NAD (N6522, Sigma-Aldrich, L'isle D'Abeau Chesne, BP 701, 38297, Saint Quentin Fallavier), and 50 μM of Prostaglandin E₂ (P4172, Sigma-Aldrich, L'isle D'Abeau Chesne, BP 701, 38297, Saint Quentin Fallavier) is prepared.

0.965 ml of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.035 mL of enzymatic suspension at 37° C. is introduced into the cuvette concomitantly with the recording (corresponding to an increase in the optical density at 340 nm). The maximum reaction rate is recorded.

The test values (containing the compounds of formula (I)) are compared with the control value (without compound of formula (I)); the results indicated represent either the percentage inhibition of the enzymatic activity of 15-PGDH for a given concentration of compound of formula (I), or the concentration at which the compound of formula (I) reduces the enzymatic activity of 15-PGDH by 50%, namely IC_{50dh}.

The results are reported in the table below.

| | | IC_{50dh} |
|---|---|---|
| | | or % |
| | | in- |
| | | hibition |
| Com- | | at |
| pound | Structure | 100 μM |
| 1 | 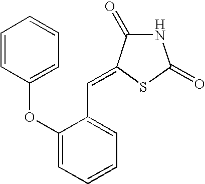 | 12 μm |
| 2 | 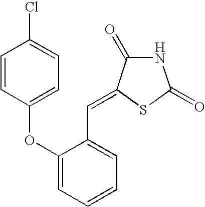 | 5 μm |
| 3 | 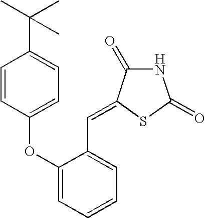 | 0.08 μm |
| 4 | 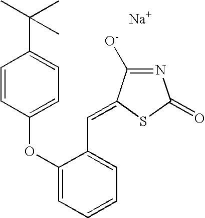 | 0.094 μm |
| 5 | 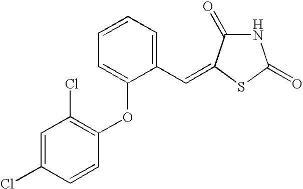 | 3 μm |
| 7 | 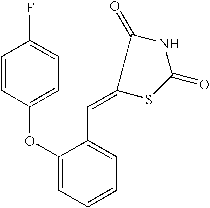 | 48% at 100 μm |
| 8 | 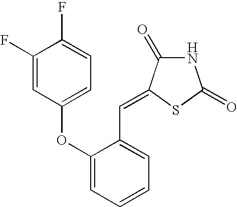 | 1.3 μm |
| 9 | 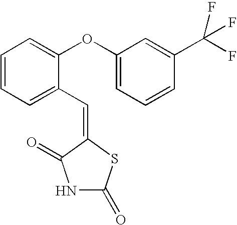 | 2.4 μm |
| 12 | 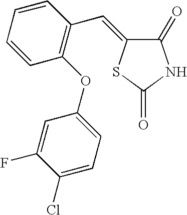 | 0.46 μm |
| 13 | 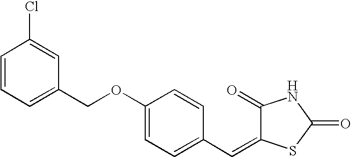 | 5 μm |
| 14 | 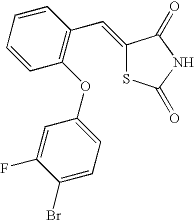 | 1.55 μm |
| 17 | 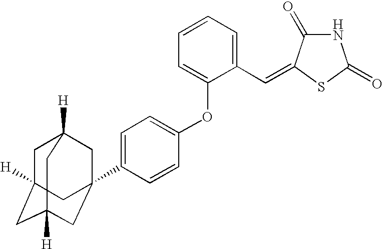 | 1.4 μm |
| 18 | 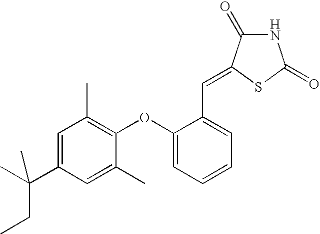 | 8 μm |
| 20 | 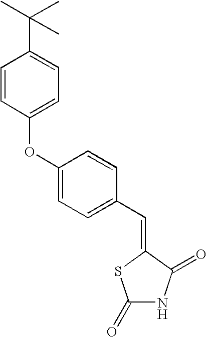 | 0.085 μm |

From this table it is clearly seen that the compounds of formula (I) are inhibitors of type-1 15-PGDH.

The compositions below are formulated via the usual techniques commonly employed in cosmetics or pharmaceutics.

### EXAMPLE 3

### Demonstration of the Effect of Compound 5 on the Tyrosinase Activity of Normal Human Melanocytes in Culture

The sodium salt of the compound (5Z)-5-[2-(4-tert-butylphenoxy)benzylidene]-1,3-thiazolidine-2,4-dione (compound 4) of structure below was tested:

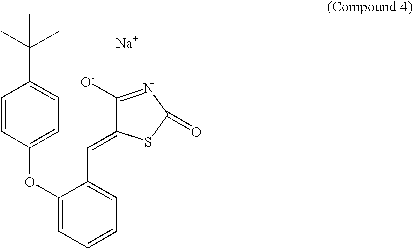

a) Melanocyte Culture Conditions:

Normal human epidermal melanocytes are cultured (9th passage) in melanocyte growth medium M2 supplemented with:

Promocell C24300 supplements

Penicillin 50 IU/ml and streptomycin 50 μg/ml (Invitrogen) under standard temperature conditions (37° C.) at 5% CO₂.

b) Preparation of the Solutions of the Test Compound:

Compound 5 is dissolved in absolute ethanol (PGE2) or in DMSO (15-PGDH inhibitors). The final titre of the prepared solutions is 10 mM. The final test concentrations range from 0.1 to 10 μM (PGE2) and 1 to 10 μM (15-PGDH inhibitors).

c)Principle of the Tests:

The tests performed consist in measuring the inhibitory effect of 15-PGDH alone or combined with PGE2* on the overall activity of tyrosinase. *PGE2 is a pigmentation modulator that is deactivated by the enzyme 15-PGDH.

The melanocytes are cultured in 96-well plates up to confluence (culture conditions described in a) and the supernatant of each of the wells is then removed and replaced with the medium containing the compounds to be evaluated.

Thus (in triplicate), the 15-PGDH inhibitor alone at 1 μM and 10 μM or, for each of these first conditions, in the presence of 0.1 μM, 1 μM or 10 μM of PGE2.

The cultures are maintained under these various conditions for 72 h, the supernatants are then removed and the cell lawns are washed with a phosphate buffer (pH=7.2). The cells are lysed with a pH 7.2 phosphate buffer containing 1% Triton X100.

The tyrosinase activity of each of the lysates is then estimated against a fungal tyrosinase range (Sigma T7755).

The methodology implemented uses L-Dopa as reaction substrate (45 minutes at 37° C.); the formation of melanin is assessed by measuring the absorbance at 450 nm.

In this test, two references are also evaluated: kojic acid (a tyrosinase inhibitor introduced at 0.1% into the culture medium) and isobutyl methyl xanthine (IBMX), a phosphodiesterase inhibitor which, by limiting the catabolism of cAMP, contributes towards increasing the overall tyrosinase activity as assessed in this test.

d) Results:

It was observed that Compound 4 (at 1 μM) has the property of stimulating the activity of tyrosinase in the presence of PGE2 (0.1 μM, 1 μM and 10 μM).

The results are as follows:

| | Test products | | | |
|---|---|---|---|---|
| | | Compound 4 | Compound 4 | Compound 4 |
| | Compound 4 | 1 μM + | 1 μM + | 1 μM + |
| | 1 μM | PGE2 0.1 μM | PGE2 1 μM | PGE2 10 μM |
| Stimulation | +16% | +27% | +40% | +32% |
| of the | | | | |
| tyrosinase | | | | |
| activity | | | | |

PGE2 alone stimulates the activity of tyrosinase
by +3% at 0.1 μM
by +9% at 1 μM and
by +15% at 10 μM.

Compound 4 does indeed have the property of stimulating the activity of tyrosinase in melanocytes.

### EXAMPLE 4

### Hair Lotion

| | | |
|---|---|---|
| Compound (5Z)-5-[2-(4-tert-butyl-phenoxy) | 1.00 | g |
| benzylidene]-1,3-thiazolidine-2,4-dione | | |
| Propylene glycol | 30.00 | g |
| Ethyl alcohol | 40.00 | g |

| | |
|---|---|
| Water | qs 100.00 g |

This lotion is applied to the scalp, once or twice a day, at a rate of 1 ml per application, while massaging the scalp gently to make the active agent penetrate. The head of hair is then dried in open air. This lotion makes it possible to prevent and/or reduce the canities of the hair.

### EXAMPLE 5

### Hair Lotion

| | | |
|---|---|---|
| Compound (5Z)-5-[2-(4-tert-butyl-phenoxy) | 1.00 | g |
| benzylidene]-1,3-thiazolidine-2,4-dione | | |
| Ethyl alcohol | 49.00 | g |

| | |
|---|---|
| Water | qs 100.00 g |

This lotion is applied to the scalp, once or twice a day, at a rate of 1 ml per application, while massaging the scalp gently to make the active agent penetrate. The head of hair is then dried in open air. This lotion makes it possible to reduce the loss and promote regrowth of the hair. It also makes it possible to prevent and/or reduce the canities of the hair.

### EXAMPLE 6

### Wax/Water Mascara

| | |
|---|---|
| Beeswax | 6.00% |
| Paraffin wax | 13.00% |
| Hydrogenated jojoba oil | 2.00% |
| Water-soluble film-forming polymer | 3.00% |
| Triethanolamine stearate | 8.00% |
| Compound (5Z)-5-[2-(4-tert-butylphenoxy) | 1.00% |
| benzylidene]-1,3-thiazolidine-2,4-dione | |
| Black pigment | 5.00% |

| | |
|---|---|
| Preservative | qs |
| Water | qs 100.00% |

This mascara is applied to the eyelashes like a standard mascara with a mascara brush. It makes it possible to reduce the loss and to promote regrowth of the eyelashes. This mascara also makes it possible to prevent and/or reduce the canities of the eyelashes.

### EXAMPLE 7

### Hair Lotion

| | | |
|---|---|---|
| Compound (5Z)-5-[2-(4-tert-butylphenoxy) | 0.10 | g |
| benzylidene]-1,3-thiazolidine-2,4-dione | | |
| Latanoprost | 0.10 | g |
| Propylene glycol | 30.00 | g |
| Ethyl alcohol | 40.00 | g |

| | |
|---|---|
| Water | qs 100.00 g |

This lotion is applied to the scalp, once or twice a day, at a rate of 1 ml per application, while massaging the scalp gently to make the active agent penetrate. The head of hair is then dried in open air. This lotion makes it possible to reduce the loss and promote regrowth of the hair. It also makes it possible to prevent and/or reduce the canities of the hair.

### EXAMPLE 8

### Hair Lotion

| | | |
|---|---|---|
| (5E)-7-{(1R,2R,3R,5S)-3,5-Dihydroxy-2- | 0.10 | g |
| [(3R)-3-hydroxy-5-phenylpentyl]cyclo- | | |
| pentyl}hept-5-enoic acid | | |
| Compound (5Z)-5-[2-(4-tert-butylphenoxy) | 0.10 | g |
| benzylidene]-1,3-thiazolidine-2,4-dione | | |
| Propylene glycol | 30.00 | g |
| Ethyl alcohol | 40.00 | g |

| | |
|---|---|
| Water | qs 100.00 g |

This lotion is applied to the scalp, once or twice a day, at a rate of 1 ml per application, while massaging the scalp gently to make the active agent penetrate. The head of hair is then dried in open air. This lotion makes it possible to prevent and/or reduce the canities of the hair.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for promoting and/or inducing and/or stimulating the pigmentation of mammalian keratin materials and/or preventing and/or limiting the depigmentation and/or bleaching thereof, comprising administering to a subject in need of such treatment, for such period of time as required to elicit the desired effect, a thus effective amount of at least one benzylidene-1,3-thiazolidine-2,4-dione compound having the structural formula (I): 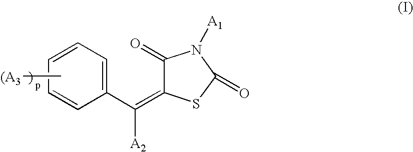
in which:
a) A₁ is:
1) a hydrogen atom; or
2) a linear or branched, saturated or unsaturated C₁-C₂₀ alkyl radical optionally substituted with one or more groups Z₁;
b) A₂ and A₃ independently are:
1) a hydrogen atom;
2) a linear or branched, saturated or unsaturated C₁-C₂₀ alkyl radical optionally substituted with one or more groups Z₁;
3) a group Z₁; with the provisos that when A₃ is Z₁, then
(i) Z₁ is other than chlorine; and
(ii) when Z₁ is OZ₂, then Z₂ is other than hydrogen, a 2-chloro-6-fluorobenzyl group, a 1,1,3,3-tetramethylbutylphenyl group and a 2,4-di(tert-butyl)phenyl group;
c) p ranges from 0 to 5 inclusive and, when n>1, the substituents A₅ may be identical or different;
d) Z₁ is:
1) a halogen; or
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂COZ′₂, NZ₂CONZ′₂Z″₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ and Si(OZ₂)(OZ′₂)OZ″₂;
e) the radicals Z₂, Z′₂, Z″₂ and Z′″₂ independently are:
1) a hydrogen atom;
2) a linear or branched, saturated or unsaturated C₁-C₂₀ alkyl radical optionally substituted with one or more groups Z₃; or
3) a saturated or unsaturated ring member of 4 to 15 atoms, optionally containing at least one heteroatom selected from among O, N and S, optionally fused to another ring, these rings being optionally substituted with one or more groups Z₃;
f) the radical Z₃ is:
1) a group Z₄;
2) a linear or branched, saturated or unsaturated C₁-C₂₀ alkyl radical optionally substituted with one or more groups Z₄; or
3) a saturated or unsaturated ring member of 4 to 15 atoms, optionally containing at least one heteroatom selected from among O, N and S, optionally fused to another ring, these rings being optionally substituted with one or more groups Z₄;
g) Z₄ is:
1) a halogen atom; or
2) a group selected from among CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ and Si(OZ₅)(OZ′₅)OZ″₅;
h) the radicals Z₅, Z′₅, Z″₅ and Z′″₅ independently are:
1) a hydrogen atom;
2) a linear or branched, saturated or unsaturated C₁-C₂₀ alkyl radical; or
3) a saturated or unsaturated ring member of 4 to 15 atoms, optionally containing at least one heteroatom selected from among O, N and S, optionally fused to another ring, these rings being optionally substituted with one or more CF₃, halogen or linear or branched C₁-C₂₀ alkyl radicals, or salt and/or solvate and/or cis-trans or Z/E isomer thereof.

2. The regime or regimen as defined by claim 1, wherein formula (I):
A₁ and A₂ simultaneously are hydrogen;
A₃ is a group Z₁;
Z₁ is a group OZ₂ in which Z₂ is a phenyl group optionally substituted with one or more adamantyl, CF₃, halogen or linear or branched, saturated or unsaturated C₁-C₂₀ alkyl radicals.

3. The regime or regimen as defined by claim 1, wherein said at least one benzylidene-1,3-thiazolidine-2,4-dione compound of formula (I) is selected from the following list of compounds, or salt and/or solvate and/or Z/E isomer thereof:

| Com- | |
|---|---|
| pound | Structure |
| 1 | 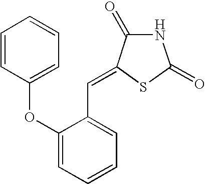 |
| 2 | 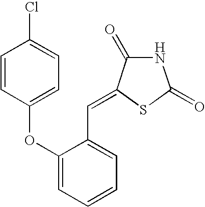 |
| 3 | 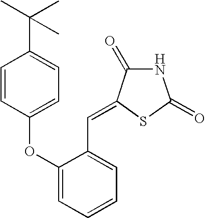 |
| 4 | 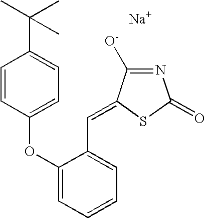 |
| 5 | 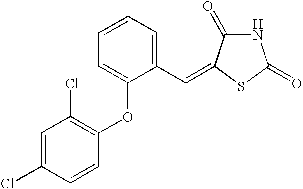 |
| 6 | 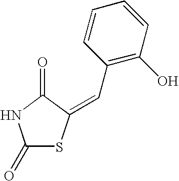 |
| 7 | 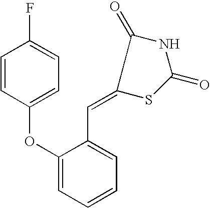 |
| 8 | 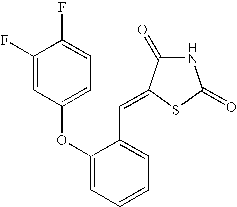 |
| 9 | 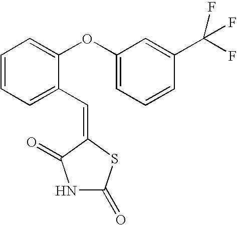 |
| 10 | 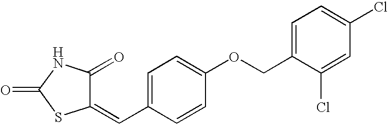 |
| 11 | 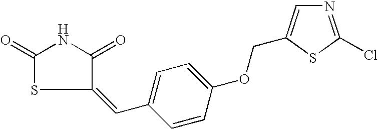 |
| 12 | 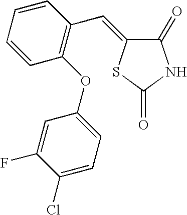 |
| 13 | 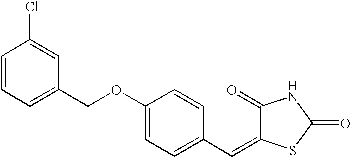 |
| 14 | 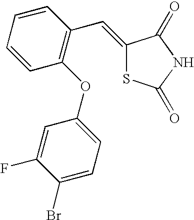 |
| 15 | 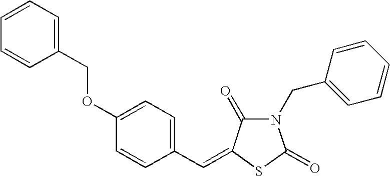 |
| 16 | 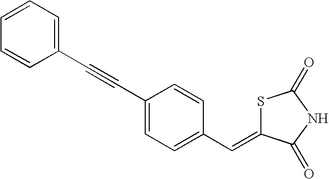 |
| 17 | 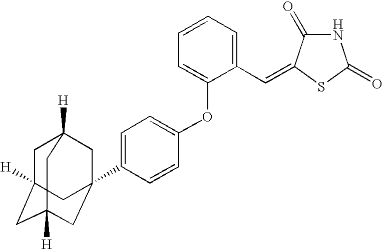 |
| 18 | 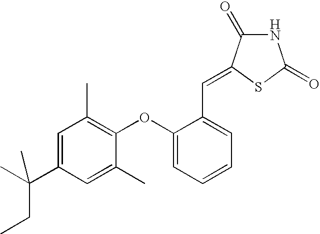 |
| 19 | 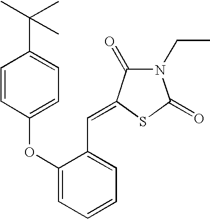 |
| 20 | 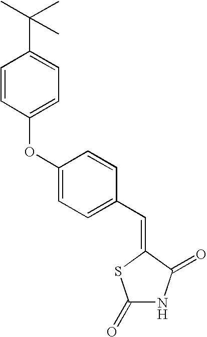 |

4. The regime or regimen as defined by claim 3, said at least one compound of formula (I) comprising:
the compound (5Z)-5-[2-(3,4-difluorophenoxy)benzylidene]-1,3-thiazolidine-2,4-dione: 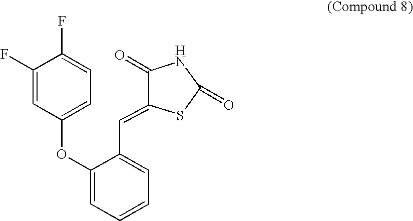
or salt and/or solvate and/or isomer thereof.

5. The regime or regimen as defined by claim 3, said at least one compound of formula (I) comprising:
the compound (5Z)-5-[2-(4-tert-butylphenoxy)benzylidene]-1,3-thiazolidine-2,4-dione of structure: 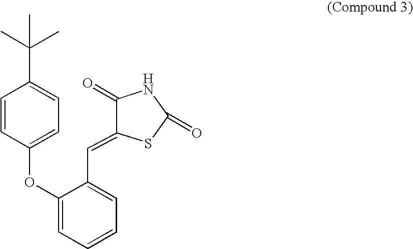
or its sodium salt of structure: 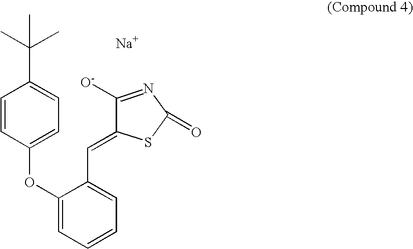
or salt and/or solvate and/or isomer thereof.

6. The regime or regimen as defined by claim 3, said at least one compound of formula (I) comprising:
the compound (5Z)-5-[2-(4-chloro-3-fluorophenoxy)benzylidene]-1,3-thiazolidine-2,4-dione (Compound 12) of structure: 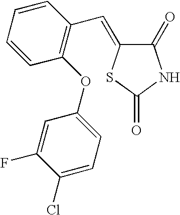
or salt and/or solvate and/or isomer thereof.

7. The regime or regimen as defined by claim 3, said at least one compound of formula (I) comprising:
the compound (5E)-5-{4-[(3-chlorobenzyl)oxy]benzylidene}-1,3-thiazolidine-2,4-dione (Compound 13) of structure: 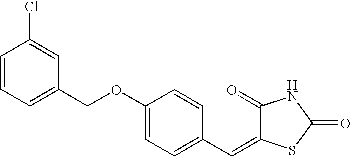
or salt and/or solvate and/or isomer thereof.

8. The regime or regimen as defined by claim 3, said at least one compound of formula (I) comprising:
the compound (5Z)-5-[4-(4-tert-butylphenoxy)benzylidene]-1,3-thiazolidine-2,4-dione (Compound 20) of structure: 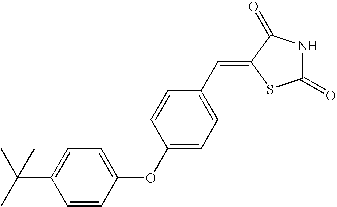
or salt and/or solvate and/or isomer thereof.

9. A regime or regimen for promoting and/or inducing and/or stimulating the pigmentation of human head hair, beard hair, moustache hair, eyelashes and/or eyebrows of an individual in need of such treatment, comprising topically applying thereon a thus effective amount of at least one benzylidene-1,3-thiazolidine-2,4-dione compound of formula (I) as defined in claim 1, or salt and/or solvate and/or isomer thereof.

10. A regime or regimen for preventing and/or limiting the depigmentation and/or whitening of human head hair, beard hair, moustache hair, eyelashes and/or eyebrows of an individual in need of such treatment, comprising topically applying thereon a thus effective amount of at least one benzylidene-1,3-thiazolidine-2,4-dione compound of formula (I) as defined in claim 1, or salt and/or solvate and/or isomer thereof.

11. A regime or regimen for preventing and/or limiting the canities of human head hair, beard hair, moustache hair, eyelashes and/or eyebrows of an individual in need of such treatment, comprising topically applying thereon a thus effective amount of at least one benzylidene-1,3-thiazolidine-2,4-dione compound of formula (I) as defined in claim 1, or salt and/or solvate and/or isomer thereof.

12. A regime or regimen for inhibiting 15-hydroxyprostaglandin dehydrogenase in a mammal in need of such treatment, comprising administering thereto a thus effective amount of at least one benzylidene-1,3-thiazolidine-2,4-dione compound as defined in Clam 1, or salt and/or solvate and/or isomer thereof.

13. A cosmetic/therapeutic composition comprising an effective pigmentation promoting/stimulating/inducing amount of at least one benzylidene-1,3-thiazolidine-2,4-dione compound as defined in claim 1, formulated into a non-toxic physiologically acceptable medium therefor.

14. The cosmetic/therapeutic composition as defined by claim 13, formulated for topical application.

15. The cosmetic/therapeutic composition as defined by claim 14, comprising from 10⁻³ to 10% of said at least one benzylidene-1,3-thiazolidine-2,4-dione compound.

16. The cosmetic/therapeutic composition as defined by claim 13, further comprising at least one prostaglandin and/or derivative thereof.
